(19) United States
(12) Patent Application Publication
(10) Pub. No.: US 2020/0283487 A1
(43) Pub. Date: Sep. 10, 2020

(54) Protein

(71) Applicants: University of Southampton, Hampshire County (GB); Spiber Technologies AB, Stockholm (SE)

(72) Inventors: Howard William Clark, Hampshire County (GB); Alastair Samuel Watson, Hampshire County (GB); Jens Madsen, Hampshire County (GB); Jan Johansson, Stockholm (SE); Anna Rising, Stockholm (SE); Nina Kronqvist, Stockholm (SE); Kerstin Nordling, Stockholm (SE)

(73) Assignees: University of Southampton, Hampshire County (GB); Spiber Technologies AB, Stockholm (SE)

(21) Appl. No.: 16/063,956

(22) PCT Filed: Dec. 21, 2016
(86) PCT No.: PCT/GB2016/054004

§ 371 Date: Jun. 19, 2018

(30) Foreign Application Priority Data
Dec. 22, 2015 (GB) 1522610.3

(51) Int. Cl. C07K 14/435; C07K 14/705

(52) U.S. Cl. CPC C07K 14/43518; A61K 38/00; C07K 2319/00; C07K 14/7056

(57) ABSTRACT

The present invention provides a fusion protein comprising: i) a solubility-enhancing moiety which is derived from the N-terminal (NT) fragment of a spider silk protein; and ii) a C-type lectin polypeptide. The present invention also provides a truncated SP-A polypeptide which lacks the N-terminal domain of full SP-A and is capable of trimerisation, and to the use of such a truncated SP-A polypeptide in treating or preventing a disease.

### FIELD OF THE INVENTION

The present invention relates to C-type lectin proteins. In particular, the present invention relates to methods for producing C-type lectin proteins, for example surfactant proteins A and D (SP-A and SP-D). In addition, the present invention provides truncated forms of SP-A and uses thereof.

### BACKGROUND TO THE INVENTION

Surfactant proteins A and D (SP-A and SP-D) are C-type lectin proteins which function as essential innate immune proteins of the lung. Both SP-A and SP-D bind to an array of different pathogens to prevent infection and to enhance clearance. In addition, SP-A and SP-D interact with numerous immune cells, allergens and other soluble components to keep the lung in a hypo-inflammatory response.

Recombinant SP-A and SP-D represent a potential therapeutic modality for a range of diseases and conditions.

By way of example, previous attempts to develop whole length recombinant SP-A have been found to be unsuitable because they were too expensive to produce, had poor functionality or were difficult to handle due to their propensity to agglomerate into higher order structures. Current attempts to provide recombinant whole length SP-D are subject to similar problems.

It has been shown that a fragment of SP-D made up of the carbohydrate recognition domain (CRD), neck and a short collagen stalk (rfhSP-D) is sufficient to maintain many of the functions of the native SP-D protein (see WO 03/03679 and WO 2015/124928). However, it has not been possible to upscale the production of rfhSP-D in a manner appropriate for subsequent development as a therapeutic using typical E. coli expression methods. For example, such production processes typically required solubilisation of the rfhSP-D polypeptide from inclusion bodies by urea treatment and subsequent refolding. This process results in low yields and, moreover, the refolding step presents a major bottle neck for the development of a viable production process for upscale manufacture.

Thus there is a need for alternative methods and approaches to produce recombinant SP-A and SP-D. In particular, there is a need for recombinant forms of SP-A and SP-D which are suitable for general therapeutic applications and for methods to produce such polypeptides.

### SUMMARY OF ASPECTS OF THE INVENTION

The present inventors have determined that the expression of a C-type lectin, for example SP-A or SP-D, as a fusion protein with a solubility-enhancing moiety which is derived from the N-terminal (NT) fragment of a spider silk protein enables the C-type lectin to be produced in a soluble form and in higher quantities than previous methods. By way of example, the use of a NT-terminal fragment of a spider silk protein fused to rfhSP-D produced greater yields than other solubility-enhancing tags, for example the maltose-binding protein (MBP) tag.

In a first aspect the present invention provides a fusion protein comprising i) a solubility-enhancing moiety which is derived from the N-terminal (NT) fragment of a spider silk protein; and ii) a C-type lectin polypeptide.

The solubility-enhancing moiety may comprise a sequence shown as SEQ ID NO: 1 to 14 or a variant thereof. The solubility-enhancing moiety may comprise a sequence shown as SEQ ID NO: 13 or a variant thereof.

The C-type lectin may be a surfactant protein A (SP-A) or a surfactant protein D (SP-D) or variant thereof having at least 70% sequence identity to SP-A or SP-D.

The SP-A polypeptide may be a truncated SP-A polypeptide. The truncated SP-A polypeptide may be a polypeptide which lacks at least amino acids 1 to 27 from the N-terminal of SEQ ID NO: 15. The truncated SP-A polypeptide may lack amino acids 1 to 30, 1 to 40, 1 to 50, 1 to 60 or 1 to 70 from the N-terminal of SEQ ID NO: 15. The truncated SP-A polypeptide may be a variant polypeptide which has at least 70% sequence identity to a truncated SP-A as described herein and is capable of trimerisation.

The truncated SP-A polypeptide may comprise SEQ ID NO: 16 or a variant of SEQ ID NO: 16 which has at least 70% sequence identity thereto and is capable of trimerisation. The truncated SP-A polypeptide may consist of SEQ ID NO: 16 or a variant of SEQ ID NO: 16 which has at least 70% sequence identity thereto and is capable of trimerisation.

The C-type lectin may be a truncated SP-D polypeptide which lacks residues 1-178 from the N-terminal of the SEQ ID NO: 17 or a variant which has at least 70% sequence identity to a truncated SP-D polypeptide which substantially lacks residues 1-178 from the N-terminal of the SEQ ID NO: 17.

The C-type lectin may be a truncated SP-D polypeptide which comprises the sequence shown in SEQ ID NO: 18, or a variant comprising an amino acid sequence having at least 70% sequence identity to the sequence shown as SEQ ID NO: 18.

The solubility-enhancing moiety may be linked directly or indirectly to the amino-terminal of the C-type lectin polypeptide.

The fusion protein may comprise a cleavage site between the solubility-enhancing moiety and the C-type lectin polypeptide. The fusion protein may comprise a purification tag.

The fusion protein may comprise the following structure:
Purification tag-solubility enhancing moiety-cleavage site-C-type lectin

The fusion protein may comprise the sequence shown as SEQ ID NO: 28 or 29.

In another aspect the present invention provides a nucleic acid sequence encoding a polypeptide according to the first aspect of the present invention.

In a further aspect the present invention provides a vector comprising a nucleic acid sequence according to the present invention.

In another aspect the present invention provides a method of producing a fusion protein according to the first aspect of the invention which comprises the steps of:
a) expressing a fusion protein according to the first aspect of the invention in a host cell;
b) obtaining a mixture including the fusion protein; and
c) optionally isolating the fusion protein.

The method may further comprising the step of isolating the C-type lectin polypeptide from the solubility-enhancing moiety.

In a further aspect the present invention provides a truncated SP-A polypeptide which lacks the N-terminal domain of full SP-A for use in treating or preventing a disease.

The truncated SP-A polypeptide may be a polypeptide which lacks at least amino acids 1 to 27 from the N-terminal of SEQ ID NO: 15. The truncated SP-A polypeptide may lack amino acids 1 to 30, 1 to 40, 1 to 50, 1 to 60 or 1 to 70 from the N-terminal of SEQ ID NO: 15. The truncated SP-A polypeptide may be a variant polypeptide which has at least 70% sequence identity to a truncated SP-A as described herein and is capable of trimerisation.

The truncated SP-A polypeptide may comprise SEQ ID NO: 16 or a variant of SEQ ID NO: 16 which has at least 70% sequence identity thereto and is capable of trimerisation. The truncated SP-A polypeptide may consist of SEQ ID NO: 16 or a variant of SEQ ID NO: 16 which has at least 70% sequence identity thereto and is capable of trimerisation.

The truncated SP-A polypeptide may comprise SEQ ID NO: 16 or a variant of SEQ ID NO: 16 which has at least 70% sequence identity thereto and is capable of trimerisation. The truncated SP-A polypeptide may consist of SEQ ID NO: 16 or a variant of SEQ ID NO: 16 which has at least 70% sequence identity thereto and is capable of trimerisation.

In a further aspect the present invention relates to a method for treating or preventing a disease which comprises the step of administering a truncated SP-A polypeptide as defined herein to a subject.

In another aspect the present invention provide the use of a truncated SP-A polypeptide as defined herein in the manufacture of a medicament for the treatment or prevention of a disease.

The disease may be an inflammatory disease such as an infection or an allergy. The disease may be a viral, bacterial or fungal infection. The disease may be an infection or an allergy.

In another aspect the present invention provides a truncated SP-A polypeptide which lacks at least amino acids 1 to 27 from the N-terminal of SEQ ID NO: 15.

The truncated SP-A polypeptide may be any truncated SP-A polypeptide as defined herein.

### DESCRIPTION OF THE FIGURES

FIG. 1—Schematic illustrating the composition of rfhSP-A and rfhSP-D relative to the native molecules

FIG. 2—Schematic illustrating A) NT-rfhSP-A and NT-rfhSP-D fusion proteins and B) subsequently generated rfhSP-A or rfhSP-D trimeric molecules

FIG. 3—Expression and purification of NT-rfhSP-A and rfhSP-A

FIG. 4—Expression and purification of NT-rfhSP-D and rfhSP-D

FIG. 5—Gel filtration of A) rfhSP-A and B), C) rfhSP-D

FIG. 6—Affinity purification of rfhSP-A and rfhSP-D and subsequent protein analysis

FIG. 7—Binding of rfhSP-A to known ligands

FIG. 8—Neutralisation of RSV by rfhSP-A

FIG. 9—Illustrative NT domains

FIG. 10—rfhSP-A binding to DerP allergen

FIG. 11—rfhSP-A binding to lipopolysaccharide

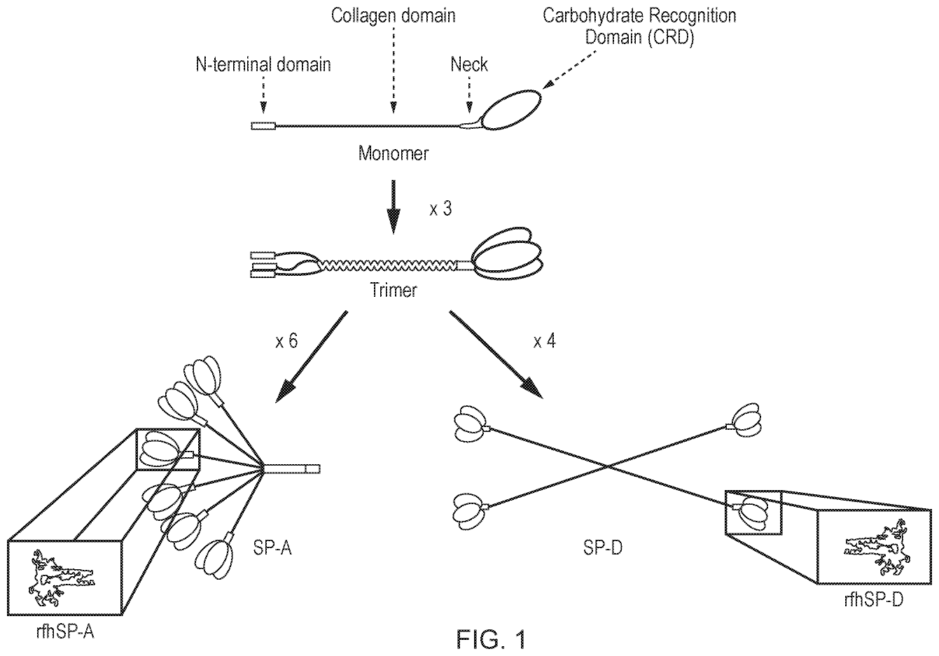

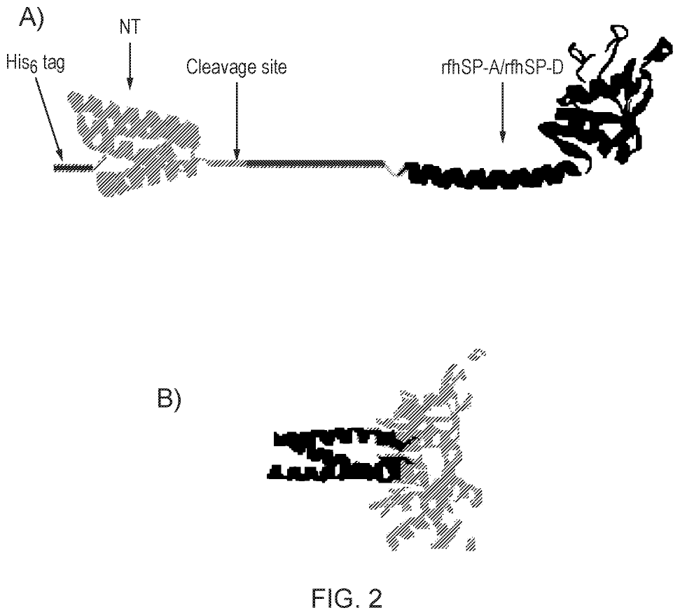

### DETAILED DESCRIPTION OF THE INVENTION

### Fusion Protein

In a first aspect the present invention provides a fusion protein.

As used herein “fusion protein” indicates that the protein is expressed from a recombinant nucleic acid, i.e. DNA or RNA that is created artificially by combining two or more nucleic acid sequences that would not normally occur together (genetic engineering). The fusion proteins according to the invention are recombinant proteins, and they are therefore not identical to naturally occurring proteins.

### Solubility-Enhancing Moiety

The solubility-enhancing moiety used in the present invention is derived from the N-terminal (NT) fragment of a spider silk protein, or spidroin. As used herein NT fragment is synonymous with the term NT domain.

Examples of spider silk proteins include major ampullate spider silks proteins (MaSp). These MaSps are generally of two types, 1 and 2. Illustrative sequences of the N-terminal (NT) fragment of a spider silk protein are shown in Table 1 below.

TABLE 1
Spidroin NT fragments

| Species and spidroin fragment | GenBank acc. no. |
|---|---|
| Euprosthenops australis MaSp 1 | AM259067 |
| Latrodectus geometricus MaSp 1 | ABY67420 |
| Latrodectus hesperus MaSp 1 | ABY67414 |
| Nephila clavipes MaSp 1 | ACF19411 |
| Argiope trifasciata MaSp 2 | AAZ15371 |
| Latrodectus geometricus MaSp 2 | ABY67417 |
| Latrodectus Hesperus MaSp 2 | ABR68855 |
| Nephila inaurata | AAZ15322 |
| madagascariensis MaSp 2 | |
| Nephila clavipes MaSp 2 | ACF19413 |
| Argiope bruennichi | BAE86855 |
| cylindriform spidroin 1 | |
| Nephila clavate | BAE54451 |
| cylindriform spidroin 1 | |
| Latrodectus Hesperus | ABD24296 |
| tubuliform spidroin | |
| Nephila clavipes | AF027972 |
| flagelliform silk protein | |
| Nephila inaurata | AF218623 |
| madagascariensis | (translated) |
| flagelliform silk protein | |

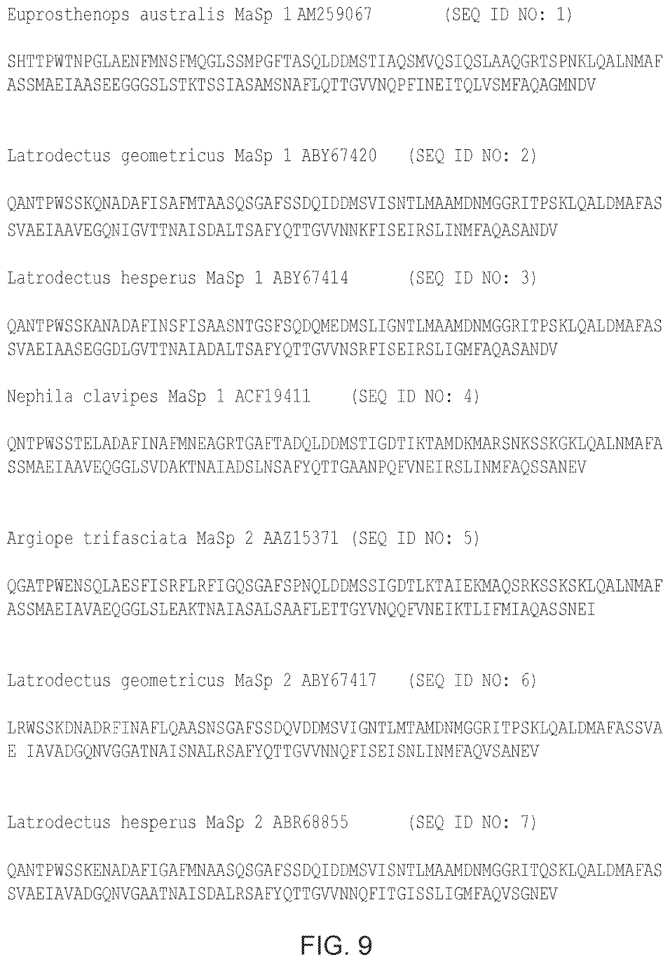

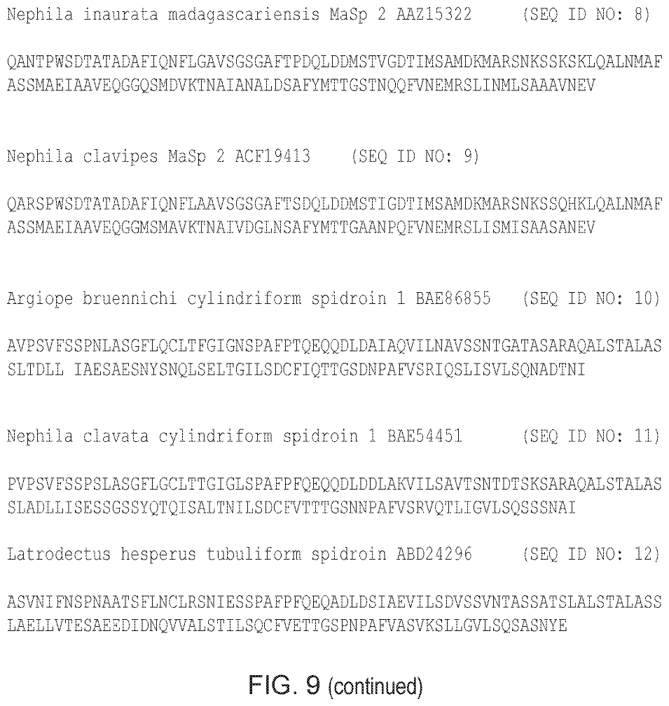

Examples of suitable NT domains are shown in FIG. 9—these sequences show the NT domain only (i.e. omitting the signal peptide).

The sequences shown in FIG. 9 constitute a consensus solubility-enhancing amino acid sequences shown as SEQ ID NO: 14. The solubility enhancing moiety may have at least 70%, at least 80%, at least 85%, at least 90%, at least 95% or at least 99% identity to SEQ ID NO: 14, provided that the sequence retains a solubility enhancing capability.

SEQ ID NO: 14
QANTPWSSPNLADAFINSF(M/L)SA(A/I)SSSGAFSADQLDDMSTIG
(D/N/Q)TLMSAMD(N/S/K)MGRSG(K/R)STKSKLQALNMAFASSMA
EIAAAESGG(G/Q)SVGVKTNAISDALSSAFYQTTGSVNPQFV(N/S)E
IRSLI(G/N)M(F/L)(A/S)QASANEV

As used herein “a solubility enhancing capability” means that the solubility of a fusion protein which includes the solubility-enhancing moiety is greater than a comparable protein which includes all the constituents of the fusion protein apart from the solubility-enhancing moiety.

For example, the fusion protein may be at least 1.5-fold, at least 2-fold, at least 5-fold, at least 10-fold, at least 20-fold, at least 50-fold, or at least 100-fold more soluble than a comparable protein which includes all the constituents of the fusion protein apart from the solubility-enhancing moiety.

The solubility of a protein may be determined by expressing the protein and subsequently comparing the amount of the protein recovered from the soluble fraction compared to the insoluble fraction. Methods for performing such a comparison are known in the art and may involve, for example, expression of the protein in a bacterial host cell (e.g. E. coli), followed by lysis/sonication of the bacterial cells and a comparison of the amount of protein present in the soluble fraction (e.g. the supernatant following centrifugation) and the insoluble fraction (e.g. the pellet following centrifugation). The comparison of the amount of protein present in each fraction may be performed using well known techniques, for example SDS-PAGE and western blotting.

The solubility enhancing moiety may be selected from any of SEQ ID NO: 1-13 or a variant thereof.

It is possible to introduce one or more mutations (substitutions, additions or deletions) into each of SEQ ID NO: 1-13 without negatively affecting its solubility enhancing capabilities. A variant of SEQ ID NO: 1-13 may, for example, have one, two or three amino acid mutations.

A variant of SEQ ID NO: 1-13 may have at least 80% sequence identity, provided that the variant sequence retains a solubility enhancing capability.

A variant of SEQ ID NO: 1-13 may have at least 85% sequence identity, provided that the variant sequence retains a solubility enhancing capability.

A variant of SEQ ID NO: 1-13 may have at least 90% sequence identity, provided that the variant sequence retains a solubility enhancing capability.

A variant of SEQ ID NO: 1-13 may have at least 95% sequence identity, provided that the variant sequence retains a solubility enhancing capability.

A variant of SEQ ID NO: 1-13 may have at least 99% sequence identity, provided that the variant sequence retains a solubility enhancing capability.

In one embodiment the solubility-enhancing moiety comprises the sequence shown as SEQ ID NO: 13 or a variant thereof which has at least 80%, at least 85%, at least 90%, at least 95% or at least 99% sequence identity.

SEQ ID NO: 13
MSHTTPWTNPGLAENFMNSFMQGLSSMPGFTASQLDKMSTIAQSMVQS
IQSLAAQGRTSPNDLQALNMAFASSMAEIAASEEGGGSLSTKTSSIAS
AMSNAFLQTTGVVNQPFINEITQLVSMFAQAGMNDVSAGNSA

Solubility-enhancing moieties such as those described herein are demonstrated in WO 2011/115538. The solubility-enhancing moiety may be any solubility-enhancing moiety as described in WO 2011/115338.

The solubility-enhancing moiety may comprise more than one solubility-enhancing moiety sequences as described herein. For example the solubility-enhancing moiety may comprise at least two, at least three or more solubility-enhancing moiety sequences as described herein.

In one embodiment, the solubility-enhancing moiety may be arranged at either the N-terminal end or the C-terminal end of the C-type lectin polypeptide. In a preferred embodiment the solubility-enhancing moiety is arranged at the N-terminal of the C-type lectin polypeptide.

C-Type Lectin Polypeptide

C-type lectin polypeptide refers to the superfamily of proteins containing C-type lectin-like domains (CTLDs). C-type lectin polypeptides include collectins, selectins, endocytic receptors, and proteoglycans. Some of these proteins are secreted and others are transmembrane proteins. They often oligomerize into homodimers, homotrimers, and higher-ordered oligomers, which increases their avidity for multivalent ligands.

The CTLD is a compact domain of 110-130 amino acid residues with a double-looped, two-stranded antiparallel β-sheet formed by the amino- and carboxy-terminal residues connected by two α-helices and a three-stranded antiparallel β-sheet. The carbohydrate recognition domain (CRD) has two highly conserved disulfide bonds and up to four sites for binding Ca²⁺, with site occupancy depending on the lectin. Amino acid residues with carbonyl side chains are often coordinated to Ca²⁺ in the CRD, and these residues directly bind to sugars when Ca²⁺ is bound in site 2. A ternary complex may be formed between a sugar in a glycan, the Ca²⁺ ion in site 2, and amino acids within the CRD. Changes in amino acids within the CRD may alter sugar specificity. Key conserved residues that bind sugars include the “EPN” and “WND” motifs within the CRD of C-type lectins. The presence of such motifs in the CRD, along with Ca²⁺ binding in site 2 and other secondary structures (including hydrogen bond donors and acceptors flanking a conserved Pro residue in the double-loop region), allows predictions as to whether a CRD binds sugar

Examples of C-type polypeptides include, but are not limited to, surfactant protein A, surfactant protein D, mannan-binding lectin, conglutinin, asialoglycoprotein receptor, dendritic cell-specific intercellular adhesion molecule 3-grabbing nonintegrin. P-selectin, macrophage C-type lectin, dendritic-cell-associated lectin 1, L-selectin, E-selectin and CD162.

### Surfactant Protein A (Sp-A) & Surfactant Protein D (Sp-D)

The basic structure of SP-A and SP-D is organized into four regions: a cysteine containing N-terminal region, a triple-helical collagen region composed of Gly-X-Y triplets, an α-helical coiled coil neck region and a globular head region at the C-terminus consisting of a homotrimeric carbohydrate recognition domain (CRD). SP-A and SP-D each assemble as trimeric subunits of basic polypeptide chains which multimerize to varying degrees of oligomers but typically is found as a dodecamer. They are formed from the linking of four trimers by disulphide bonds at the N termini.

The carboxy-terminal domains have C-type (calcium-dependent) lectin activity that mediates the interaction with a wide variety of pathogens. This results in pathogen opsonization and enhanced uptake by phagocytes. The neck region has disulphide binding sites that form inter-chain bonds that are required for assembling the SP-A and SP-D into trimers. The N-terminal domain confers structural stability on the protein, owing to its disulphide-bonding pattern and dictates the degree of multimerization of the single trimeric subunits.

### Surfactant Protein A

In one embodiment the C-type lectin is SP-A.

SP-A is an innate immune system collectin which has collagen-like domains. It is primarily expressed in the lungs and facilitates phagocytosis by alveolar macrophages through opsonisation.

The SP-A polypeptide may be a human SP-A having the GenBank accession number NM_005411.

An amino acid sequence of such a human SP-A is shown as SEQ ID NO: 15.

SEQ ID NO: 15
MWLCPLALNLILMAASGAVCEVKDVCVGSPGIPGTPGSHGLPGRDGRD
GLKGDPGPPGPMGPPGEMPCPPGNDGLPGAPGIPGECGEKGEPGERGP
PGLPAHLDEELQATLHDFRHQILQTRGALSLQGSIMTVGEKVFSSNGQ
SITFDAIQEACARAGGRIAVPRNPEENEAIASFVKKYNTYAYVGLTEG
PSPGDFRYSDGTPVNYTNWYRGEPAGRGKEQCVEMYTDGQWNDRNCLY
SRLTICEF

The amino acid sequence of SP-A may be lacking the signal sequence (e.g. the amino acid sequence may lack residues 1 to 20 of SEQ ID NO: 15).

The SP-A polypeptide may be a fragment or variant of SP-A.

In a preferred embodiment, the SP-A fragment is a truncated SP-A polypeptide which lacks at least amino acids 1 to 27 from the N-terminal of SEQ ID NO: 15. The truncated SP-A polypeptide may be a variant which has at least 70% sequence identity to a polypeptide which lacks at least amino acids 1 to 27 from the N-terminal of SEQ ID NO: 15 and is capable of trimerisation. In other words, the truncated SP-A polypeptide or variant thereof lacks the N-terminal domain of native human SP-A (e.g. as shown in SEQ ID NO: 15) but is capable of trimerisation.

In one embodiment, the truncated SP-A is capable of functional trimerisation. That is, the truncated SP-A polypeptide is capable of stable trimerisation in the absence of a cross-linking agent and is capable of binding to ligands which the full length SP-A polypeptide is capable of binding.

The truncated SP-A polypeptide may lack amino acids 1 to 30, 1 to 40, 1 to 50, 1 to 60 or 1 to 70 from the N-terminal of SEQ ID NO: 15. The truncated SP-A polypeptide may be a variant which has at least 70% sequence identity to a polypeptide which lacks 1 to 30, 1 to 40, 1 to 50, 1 to 60 or 1 to 70 from the N-terminal of SEQ ID NO: 15 and is capable of trimerisation.

The truncated SP-A polypeptide comprises at least a head region, a neck region and a Gly-Xaa-Yaa motif.

The amino acid sequence of the head region of native human SP-A is shown as SEQ ID NO: 30.

(SEQ ID NO: 30)
SIMTVGEKVFSSNGQSITFDAIQEACARAGGRIAVPRNPEENEAIASF
VKKYNTYAYVGLTEGPSPGDFRYSDGTPVNYTNWYRGEPAGRGKEQCV
EMYTDGQWNDRNCLYSRLTICEF

The head region may comprise the sequence shown as SEQ ID NO: 30 or a variant thereof which has at least 70, 80, 85, 90, 95 or 99% sequence identity to SEQ ID NO: 30.

The amino acid sequence of the neck region of native human SP-A is shown as SEQ ID NO: 31.

(SEQ ID NO: 31)
AHLDEELQATLHDFRHQILQTRGALSLQG

The neck region may comprise the sequence shown as SEQ ID NO: 31 or a variant thereof which has at least 70, 80, 85, 90, 95 or 99% sequence identity to SEQ ID NO: 31.

The Gly-Xaa-Yaa motif comprises at least seven Gly-Xaa-Yaa repeats. In a preferred embodiment, the Gly-Xaa-Yaa motif comprises the sequence shown as SEQ ID NO: 32

(SEQ ID NO: 32)
GPGIPGECGEKGEPGERGPPGLP.

The truncated SP-A polypeptide may comprise SEQ ID NO: 16 or a variant thereof which has at least 70% sequence identity and is capable of trimerisation.

In one embodiment the truncated SP-A polypeptide may consist of SEQ ID NO: 16 or a variant thereof which has at least 70% sequence identity and is capable of trimerisation.

SEQ ID NO: 16
GPGIPGECGEKGEPGERGPPGLPAHLDEELQATLHDFRHQILQTRGAL
SLQGSIMTVGEKVFSSNGQSITFDAIQEACARAGGRIAVPRNPEENEA
IASFVKKYNTYAYVGLTEGPSPGDFRYSDGTPVNYTNWYRGEPAGRGK
EQCVEMYTDGQWNDRNCLYSRLTICEF

Surfactant Protein D (SP-D)

In one embodiment the C-type lectin is SP-D.

SP-D is 46 kDa hydrophilic calcium dependent, carbohydrate binding protein, classified under the collectin family of proteins. It is encoded by the long arm of human chromosome 10.

SP-D is secreted by Alveolar Epithelial Type II cells (ATII) cells, sub mucosal cells and Clara cells. It has its own secretory vesicle that extrudes from ATII cells into the alveolar lumen and associates with the underlying hydrophilic layer. Although the majority of SP-D is expressed in the lung, transcripts of SP-D have also been detected in other parts of the body, such as the intestine, thymus, prostrate, brain, testes, salivary gland, lachrymal gland and heart.

In a steady state, SP-D has important functions in maintaining the surfactant homeostasis and normal physiology of the lung. SP-D enhances clearance and uptake of apoptotic cells by binding to cell debris and cell-surface DNA, thereby controlling inflammation, also plays an essential role for maintaining immunological homeostasis in the lung.

SP-D can directly bind to host immune cells and influence their response and phagocytic activity. SP-D displays chemotactic activity on neutrophils and certain mononuclear phagocytes and can induce directional actin polymerization in alveolar macrophages in a concentration dependent manner. It also modulates the production of cytokines and inflammatory mediators in a pathogen dependent manner.

Surfactant proteins (including SP-D) have also been shown to play protective role against lung infection, allergy, asthma and inflammation.

SP-D may refer to human SP-D, for example, the sequences disclosed in the above references, or in GenBank accession numbers NM_003019.1, XM_005776.2, X65018.1 and L05485.1.

The SP-D may be a human SP-D having the GenBank accession number NM_003019.1. The amino acid of such a human SP-D is shown as SEQ ID NO: 17.

SEQ ID NO: 17
MLLFLLSALVLLIQPLGYLEAEMKTYSHRTMPSACILVMCSSVESGLP
GRDGRDGREGPRGEKGDPGLPGAAGQAGMPGQAGPVGPKGDNGSVGEP
GPKGDIGPSGPPGPPGVPGPAGREGALGKQGNIGPQGKPGPKGEAGPK
GEVGAPGMQGSAGARGLAGPKGERGVPGERGVPGNTGAAGSAGAMGPQ
GSPGARGPPGLKGDKGIPGDKGAKGESGLPDVASLRQQVEALQGQVQH
LQAAFSQYKKVELFPNGQSVGEKIFKIAGFVKPFTEAQLLCTQAGGQL
ASPRSAAENAALQQLVVAKNEAAFLSMIDSKIEGKFIYPISESLVYSN
WAPGEPNDDGGSEDCVEIFINGKWNDRACGEKRLVVCEF

The amino acid sequence of SP-D may be lacking the signal sequence (e.g. the amino acid sequence may lack residues 1 to 20 of SEQ ID NO: 17).

The SP-D polypeptide may be a fragment or variant of SP-D.

In a preferred embodiment, the SP-D fragment is a truncated SP-D polypeptide which substantially lacks residues 1-178 from the N-terminal of the SEQ ID NO: 17. The SP-D polypeptide may be a variant which has at least 70% sequence identity to a truncated SP-D polypeptide which substantially lacks residues 1-178 from the N-terminal of the SEQ ID NO: 17 and is capable of trimerisation. The SP-D polypeptide may be a recombinant fragment of SP-D, preferably human SP-D sequence shown in SEQ ID NO: 17, comprising substantially residues 179-355 of the sequence shown as SEQ ID NO: 17.

The truncated SP-D polypeptide comprises at least a head region, a neck region and a Gly-Xaa-Yaa motif.

The amino acid sequence of the head region of native human SP-A is shown as SEQ ID NO: 33.

(SEQ ID NO: 33)
NGQSVGEKIFKTAGFVKPFTEAQLLCTQAGGQLASPRSAAENAALQQL
VVAKNEAAFLSMTDSKTEGKFTYPTGESLVYSNWAPGKPNDDGGSEDC
VEIFTNGKWNDRACGEKRLVVCEF

The head region may comprise the sequence shown as SEQ ID NO: 33 or a variant thereof which has at least 70, 80, 85, 90, 95 or 99% sequence identity to SEQ ID NO: 33.

The amino acid sequence of the neck region of native human SP-A is shown as SEQ ID NO: 34.

(SEQ ID NO: 34)
DVASLRQQVEALQGQVQHLQAAFSQYKKVELFP

The neck region may comprise the sequence shown as SEQ ID NO: 34 or a variant thereof which has at least 70, 80, 85, 90, 95 or 99% sequence identity to SEQ ID NO: 34.

The Gly-Xaa-Yaa motif comprises at least seven Gly-Xaa-Yaa repeats. In a preferred embodiment, the Gly-Xaa-Yaa motif comprises the sequence shown as SEQ ID NO: 35

(SEQ ID NO: 35)
GPGLKGDKGIPGDKGAKGESGLP.

The sequence of such a SP-D fragment was previously disclosed in WO 03/035679 and is shown herein as SEQ ID NO: 18 (rfhSP-D).

SEQ ID NO: 18
GPGLKGDKGIPGDKGAKGESGLPDVASLRQQVEALQGQVQHLQAAFSQ
YKKVELFPNGQSVGEKIFKTAGFVKPFTEAQLLCTQAGGQLASPRSAA
ENAALQQLVVAKNEAAFLSMTDSKTEGKFTYPTGESLVYSNWAPGKPN
DDGGSEDCVEIFTNGKWNDRACGEKRLVVCE

The SP-D polypeptide may comprise SEQ ID NO: 18 or a variant which has at least 70% sequence identity thereto and is capable of trimerisation.

In one embodiment the SP-D polypeptide may consist of SEQ ID NO: 18 or a variant which has at least 70% sequence identity thereto and is capable of trimerisation.

### Variant

As used herein, a variant sequence is taken to include an amino acid sequence which is at least 70, 80, 85, 90, 95, 98 or 99% identical, preferably at least 95 or 99% identical to a sequence shown herein, for example SP-A or SP-D or a fragment thereof.

A variant may be an amino acid sequence which is at least 70% identical to a sequence shown herein, for example SP-A or SP-D or a fragment thereof.

A variant may be an amino acid sequence which is at least 80% identical to a sequence shown herein, for example SP-A or SP-D or a fragment thereof.

A variant may be an amino acid sequence which is at least 85% identical to a sequence shown herein, for example SP-A or SP-D or a fragment thereof.

A variant may be an amino acid sequence which is at least 90% identical to a sequence shown herein, for example SP-A or SP-D or a fragment thereof.

A variant may be an amino acid sequence which is at least 95% identical to a sequence shown herein, for example SP-A or SP-D or a fragment thereof.

A variant may be an amino acid sequence which is at least 98% identical to a sequence shown herein, for example SP-A or SP-D or a fragment thereof.

A variant may be an amino acid sequence which is at least 99% identical to a sequence shown herein, for example SP-A or SP-D or a fragment thereof.

Although a variant can also be considered in terms of similarity (i.e. amino acid residues having similar chemical properties/functions), in the context of the present invention it is preferred to express a variant in terms of sequence identity.

Sequence comparisons can be conducted by eye, or more usually, with the aid of readily available sequence comparison programs. These publicly and commercially available computer programs can calculate sequence identity between two or more sequences.

Sequence identity may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % sequence identity therefore firstly requires the production of an optimal alignment, taking into consideration gap penalties. A suitable computer program for carrying out such an alignment is the GCG Wisconsin Bestfit package (University of Wisconsin, U.S.A; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program.

Although the final sequence identity can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % sequence identity. The software typically does this as part of the sequence comparison and generates a numerical result.

The terms “variant” according to the present invention includes any substitution of, variation of, modification of, replacement of, deletion of or addition of one (or more) amino acids from or to the sequence providing the resultant amino acid sequence retains substantially the same activity as the unmodified sequence.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Linker

The solubility-enhancing moiety of the present fusion protein may be linked directly or indirectly to the C-type lectin polypeptide. A direct linkage implies a direct covalent binding between the solubility-enhancing moiety and the C-type lectin polypeptide without an intervening linker sequence.

An indirect linkage implies that the solubility-enhancing moiety and the C-type lectin polypeptide are linked by intervening amino acid sequences, such are a linker sequences and/or one or more further solubility-enhancing moiety.

Cleavage Site

In one embodiment, the linker sequence is an amino acid sequence which provides a cleavage site.

The cleavage site may be any sequence which enables the solubility-enhancing moiety and the C-type lectin polypeptide to be separated.

By way of example the cleavage site may be a Human Rhinovirus 3C Protease cleavage site, an Enterokinase (EKT) cleavage site, a Factor Xa (FXa) cleavage site, a Tobacco Etch Virus Protease (TEV) cleavage site or a Thrombin (Thr) cleavage site. Each of these cleavage sites provides an amino acid sequence which acts as a recognition sequence to target the action of a specific protease activity.

HRV is a highly specific protease that cleaves between the Glu and Gly residues in the cleavage tag. EKT is an intestinal enzyme normally involved in the protease cleavage of Trypsin. It cleaves after the Lys in is recognition sequence. Factor Xa cleaves after the Arg residue but can also cleave less frequently at secondary basic sites. TEV protease is a highly sequence-specific cysteine protease which is chymotrypsin-like proteases. It is very specific for its target cleavage site and is therefore frequently used for the controlled cleavage of fusion proteins both in vitro and in vivo. Thr is a serine protease.

The sequence of each of these cleavage sites are as follows: Human Rhinovirus 3C: LEVLFQ/GP (SEQ ID NO: 19); EKT: DDDDK/(SEQ ID NO: 20); FXa: IEGR/(SEQ ID NO: 21); TEV: ENLYFQ/G (SEQ ID NO: 22) and Thr: LVPR/GS (SEQ ID NO: 23)

In each of the above cleavage site sequences “/” represents the specific site where cleavage occurs.

Purification Tag

The present fusion protein may comprise a purification tag which enables the fusion protein to be isolated during a production method as described herein.

By way of example, the purification tag may comprise a histidine tag, a hemagglutinin (HA), a V5 or a myc tag.

A histidine tag is an amino acid motif in proteins that consists of at least six histidine (His) residue. Proteins comprising a histidine tag may be purified using an affinity medium comprising nickel or cobalt using techniques which are known in the art. An example of a histidine tag amino acid sequence is MGHHHHHH (SEQ ID NO: 24).

Hemagglutinin (HA) is a surface glycoprotein of influenza which is required for the infectivity of the human virus. The HA tag is derived from the HA-molecule corresponding to amino acids 98-106. Proteins comprising a HA tag may be purified using an affinity medium comprising an anti-HA antibody using techniques which are known in the art. The HA tag may comprise the sequence shown as YPYDVPDYA (SEQ ID NO: 25).

The V5 epitope tag (V5) is derived from a small epitope (Pk) present on the P and V proteins of the paramyxovirus of simian virus 5 (SV5). Proteins comprising a V5 tag may be purified using an affinity medium comprising an anti-V5 antibody using techniques which are known in the art. The V5 tag may comprise the sequence shown as SEQ ID NO: GKPIPNPLLGLDST (SEQ ID NO: 36) or IPNPLLGLD (SEQ ID NO: 26).

A myc tag is a polypeptide protein tag derived from the c-myc gene product. Proteins comprising a myc tag may be purified using an affinity medium comprising an anti-myc antibody using techniques which are known in the art. The MYC tag may comprise the sequence shown as EQKLISEEDL (SEQ ID NO: 27).

The purification tag may be arranged at either the N-terminal end or the C-terminal end of the fusion protein. In one embodiment the purification tag is arranged at the N-terminal of the fusion protein.

Structure

In one embodiment the fusion protein of the present invention comprises the following structure: Purification tag—solubility enhancing moiety—cleavage site—C-type lectin.

The purification tag, solubility enhancing moiety, cleavage site and C-type lectin may be any such entity as described herein.

In one embodiment the fusion protein comprises the sequence shown as SEQ ID NO: 28.

SEQ ID NO: 28
MGHHHHHHMSHTTPWTNPGLAENFMNSFMQGLSSMPGFTASQLDKMST
IAQSMVQSIQSLAAQGRTSPNDLQALNMAFASSMAEIAASEEGGGSLS
TKTSSIASAMSNAFLQTTGVVNQPFINEITQLVSMFAQAGMNDVSAGN
SALEVLFQGPGIPGECGEKGEPGERGPPGLPAHLDEELQATLHDFRHQ
ILQTRGALSLQGSIMTVGEKVFSSNGQSITFDAIQEACARAGGRIAVP
RNPEENEAIASFVKKYNTYAYVGLTEGPSPGDFRYSDGTPVNYTNWYR
GEPAGRGKEQCVEMYTDGQWNDRNCLYSRLTICEF
Italic-His purification tag
Standard-Solubility enhancing moiety
Underline-cleavage site
Bold-rfhSP-A

In one embodiment the fusion protein comprises the sequence shown as SEQ ID NO: 29.

SEQ ID NO: 29
MGHHHHHHMSHTTPWTNPGLAENFMNSFMQGLSSMPGFTASQLDKMST
IAQSMVQSIQSLAAQGRTSPNDLQALNMAFASSMAEIAASEEGGGSLS
TKTSSIASAMSNAFLQTTGVVNQPFINEITQLVSMFAQAGMNDVSAGN
SALEVLFQGPGLKGDKGIPGDKGAKGESGLPDVASLRQQVEALQGQVQ
HLQAAFSQYKKVELFPNGQSVGEKIFKTAGFVKPFTEAQLLCTQAGGQ
LASPRSAAENAALQQLVVAKNEAAFLSMTDSKTEGKFTYPTGESLVYS
NWAPGKPNDDGGSEDCVEIFTNGKWNDRACGEKRLVVCEF
Italic-His purification tag
Standard-Solubility enhancing moiety
Underline-cleavage site
Bold-rfhSP-D

Nucleic Acid

In another aspect the present invention provides a nucleic acid encoding a fusion protein of the first aspect of the present invention.

As used herein, the terms “polynucleotide”, “nucleotide”, and nucleic acid are intended to be synonymous with each other. “Polynucleotide” generally refers to any polyribonucleotide or polydeoxribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Vector

In another aspect the present invention provides a vector which comprises a nucleic acid sequence according to the present invention. Such a vector may be used to introduce the nucleic acid sequence into a host cell so that the host cell expresses a fusion protein according to the first aspect of the invention.

The vector may, for example, be a plasmid or a viral vector. In one embodiment the vector is a plasmid.

The term plasmid covers any DNA transcription unit comprising a nucleic acid according to the invention and the elements necessary for its in vivo expression in a desired cell; and, in this regard, it is noted that a supercoiled or non-supercoiled, circular plasmid, as well as a linear form, are intended to be within the scope of the invention.

Method

In one aspect the present invention relates to a method of producing a fusion protein according to the present invention which comprises the steps of: a) expressing a fusion protein according to first aspect of the present invention in a host cell; b) obtaining a mixture including the fusion protein; and c) optionally isolating the fusion protein.

The fusion protein may be expressed in a host cell using standard techniques which are known in the art.

For example, the fusion protein may be expressed by introducing a nucleic acid sequence or a vector as described herein into a host cell such that the fusion protein is expressed by the host cell. The nucleic acid sequence or vector may be introduced using standard techniques which are appropriate for the particular host cell. By way of example, the nucleic acid or vector may be introduced using standard techniques such as transformation or standard transfection techniques, such as electroporation or lipofection.

The host cell may be any suitable cell type, for example a bacteria or eukaryotic cell (e.g. a yeast, insect or mammalian cell). In a preferred embodiment the host cell is a bacterial cell, for example an E. coli cell.

Once the nucleic acid or vector is introduced into the host cell, the host cell is cultured in standard culture conditions which are appropriate for the particular host cell.

A mixture including the fusion protein may be obtained, for example, by lysing or mechanically disrupting the host cell. The mixture may also be obtained by collecting the cell culture medium, if the fusion protein is secreted by the host cell. The fusion protein can therefore be obtained by standard techniques.

The mixture may be subjected to centrifugation, gel filtration, chromatography, dialysis or any other suitable technique to separate the mixture.

The method may comprise the step isolating the fusion protein. This step may be performing by any suitable technique or method, for example, by affinity chromatography, ion exchange chromatography.

In a preferred embodiment, the fusion protein is isolated by affinity chromatography/purification using an affinity medium which binds to a purification tag as described herein.

The method may further comprise the step of isolating the C-type lectin polypeptide from the solubility-enhancing moiety. In a preferred embodiment, the C-type lectin polypeptide is isolated by cleavage of a cleavage site as described herein.

Standard techniques suitable for performing the present method are well known in the art (see, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wley & Sons; each of which is herein incorporated by reference.

Treating a Disease

In another aspect, the present invention provides a truncated SP-A polypeptide which lacks the N-terminal domain of full SP-A for use in treating or preventing a disease.

Full length SP-A typically forms hexatrimer multimers with characteristic “bouquet-like” structures. This is in contrast to full length SP-D, which typically forms higher order oligomer structures with a characteristic “cruciform-like” shape. Further, the antigen binding specificities and mechanisms of action of SP-A and SP-D may be different when binding to hydroxyl groups on surface carbohydrates, lipid (e.g. lipid A of LPS) and proteins (e.g SP-A interacts differently with apoptotic cells and has different candidate receptor molecules) (see for example, Ng et al.; 2012; Journal of Biomedicine and Biotechnology; Article ID 732191). Accordingly, it is known that there are differences between the mechanisms of action between SP-A and SP-D. Without wishing to be bound by theory, these differences may be caused by different patterns of charge distribution on the trimeric molecules and the difference shape of the trimeric head groups of SP-A and SP-D, respectively.

In another aspect, the present invention relates to a method for treating or preventing a disease which comprises the step of administering a truncated SP-A polypeptide as described herein to a subject.

In a further aspect the present invention provides the use of a truncated SP-A polypeptide as described herein in the manufacture of a medicament for the treatment or prevention of a disease.

The term treat/treatment/treating refers to administering a truncated SP-A as described herein to a subject having an existing disease or condition in order to lessen, reduce or improve at least one symptom associated with the disease and/or to slow down, reduce or block the progression of the disease.

The term prevent/prevention/preventing means to administer a truncated SP-A as described herein to a subject who is not showing any symptoms of a disease as described herein to reduce or prevent development of at least one symptom associated with the disease.

The truncated SP-A polypeptide may be a truncated SP-A polypeptide as described herein.

The disease may be an inflammatory disease such as an infection or an allergy. The inflammatory disease may be an inflammatory lung disease.

Examples of inflammatory lung diseases include COPD, asthma, cystic fibrosis, bacterial infection, viral infection, fungal infection, allergy, neonatal chronic lung disease, neonatal respiratory distress syndrome (RDS), adult respiratory distress syndrome, pulmonary fibrosis, emphysema, interstitial inflammatory lung disease, sarcoidosis, pneumonia, chronic inflammatory lung disease and neonatal chronic inflammatory lung disease.

The disease may be an infection or an allergy.

The infection may be a viral, a bacterial or a fungal infection.

In one embodiment the infection is a viral infection. Suitably, the viral infection may be a viral infection of the lung(s), for example respiratory syncytial virus (RSV), influenza A, influenza B, coronavirus, rhinovirus, parainfluenza virus or adenovirus infection. In one particular embodiment the infection is a respiratory syncytial virus (RSV) infection. The present inventors have shown that a truncated SP-A molecule as defined herein was more effective at reducing RSV infection than native SP-A. This was surprising as the SP-A N-terminal domain and subsequent oligomeric domain has previously been considered important for its anti-pathogenic function, particularly in trapping microbes in the net-like surfactant tubular myelin structure formed by SP-A Ikegami et al.; 2001; J Biol Chem; 276; 38542-38548 & Ng et al.; 2012; Journal of Biomedicine and Biotechnology; Article ID 732191).

In one embodiment the infection is a bacterial infection. Suitably, the bacterial infection may be a bacterial infection of the lung(s), for example Streptococcus pneumoniae, Haemophilus species, Staphylococcus aureus and Mycobacterium tuberculosis infection.

In one embodiment the infection is a fungal infection. Suitably, the fungal infection may be a fungal infection of the lung(s), for example Aspergillus infection.

In one embodiment the allergy may be caused by a respiratory allergen. In one embodiment the allergy may be a dust mite allergy, cat allergy (e.g. cat dander allergy), dog allergy (e.g. dog dander allergy), pollen allergy.

In one embodiment the present invention provides a truncated SP-A polypeptide which comprises or consists of SEQ ID NO: 16 or a variant thereof which has at least 70% sequence identity and is capable of trimerisation for use in treating or preventing a disease.

In one embodiment the present invention provides a truncated SP-A polypeptide which comprises or consists of SEQ ID NO: 16 or a variant thereof which has at least 70% sequence identity and is capable of trimerisation for use in treating or preventing an infection or an allergy.

In one embodiment the present invention provides a truncated SP-A polypeptide which comprises or consists of SEQ ID NO: 16 or a variant thereof which has at least 70% sequence identity and is capable of trimerisation for use in treating or preventing a viral, a bacterial or a fungal infection.

In one embodiment the present invention provides a truncated SP-A polypeptide which comprises or consists of SEQ ID NO: 16 or a variant thereof which has at least 70% sequence identity and is capable of trimerisation for use in treating or preventing a viral infection.

In one embodiment the present invention provides a truncated SP-A polypeptide which comprises or consists of SEQ ID NO: 16 or a variant thereof which has at least 70% sequence identity and is capable of trimerisation for use in treating or preventing a bacterial infection.

In one embodiment the present invention provides a truncated SP-A polypeptide which comprises or consists of SEQ ID NO: 16 or a variant thereof which has at least 70% sequence identity and is capable of trimerisation for use in treating or preventing a fungal infection.

In one embodiment the infection is not a parasitic infection, in particular the infection is not a parasitic nematode infection.

Administration

The administration of a truncated SP-A can be accomplished using any of a variety of routes that make the active ingredient bioavailable. For example, the truncated SP-A can be administered by oral and parenteral routes, intranasally, intraperitoneally, intravenously, subcutaneously, transcutaneously or intramuscularly.

Preferably, truncated SP-A is administered such that it is available in an active form in the lungs of the subject to which it is administered.

For example, the truncated SP-A may be administered intranasally or in the form of an aerosol.

Typically, a physician will determine the actual dosage that is most suitable for an individual subject and it will vary with the age, weight and response of the particular patient. The dosage is such that it is sufficient to reduce and/or prevent disease symptoms.

The dosage is such that it is sufficient to stabilise or improve symptoms of the disease.

Pharmaceutical Composition

The present invention also provides a pharmaceutical composition comprising a truncated SP-A polypeptide for use in the treatment and/or prevention of a disease as described herein.

The pharmaceutical composition comprises a truncated SP-A as defined herein.

The pharmaceutical composition comprises a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may comprise as (or in addition to) the carrier, excipient or diluent, any suitable binder(s), lubricant(s), suspending agent(s), coating agent(s), solubilising agent(s) and other carrier agents.

Definitions of terms appear throughout the specification. Before the exemplary embodiments are described in more detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, N.Y. (1991) provide one of skill with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “an enzyme” includes a plurality of such candidate agents and equivalents thereof known to those skilled in the art, and so forth.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of also include the term” consisting of′.

The invention will now be further described by way of Examples, which are meant to serve to assist one of ordinary skill in the art in carrying out the invention and are not intended in any way to limit the scope of the invention.

### EXAMPLES

### Example 1—Expression and Purification of rfhSP-A Using a Solubility-Enhancing Moiety from the N-Terminal (NT) Fragment of a Spider Silk Protein

rfhSP-A with an N-terminal tag derived from spider silk protein (SEQ ID NO: 28) was successfully expressed in E. coli and purified with successful subsequent purification of rfhSP-A.

SEQ ID NO: 28
MGHHHHHHMSHTTPWTNPGLAENFMNSFMQGLSSMPGFTASQLDKMST
IAQSMVQSIQSLAAQGRTSPNDLQALNMAFASSMAEIAASEEGGGSLS
TKTSSIASAMSNAFLQTTGVVNQPFINEITQLVSMFAQAGMNDVSAGN
SALEVLFQGPGIPGECGEKGEPGERGPPGLPAHLDEELQATLHDFRHQ
ILQTRGALSLQGSIMTVGEKVFSSNGQSITFDAIQEACARAGGRIAVP
RNPEENEAIASFVKKYNTYAYVGLTEGPSPGDFRYSDGTPVNYTNWYR
GEPAGRGKEQCVEMYTDGQWNDRNCLYSRLTICEF
Italic-His purification tag
Standard-Solubility enhancing moiety
Underline-cleavage site
Bold-rfhSP-A

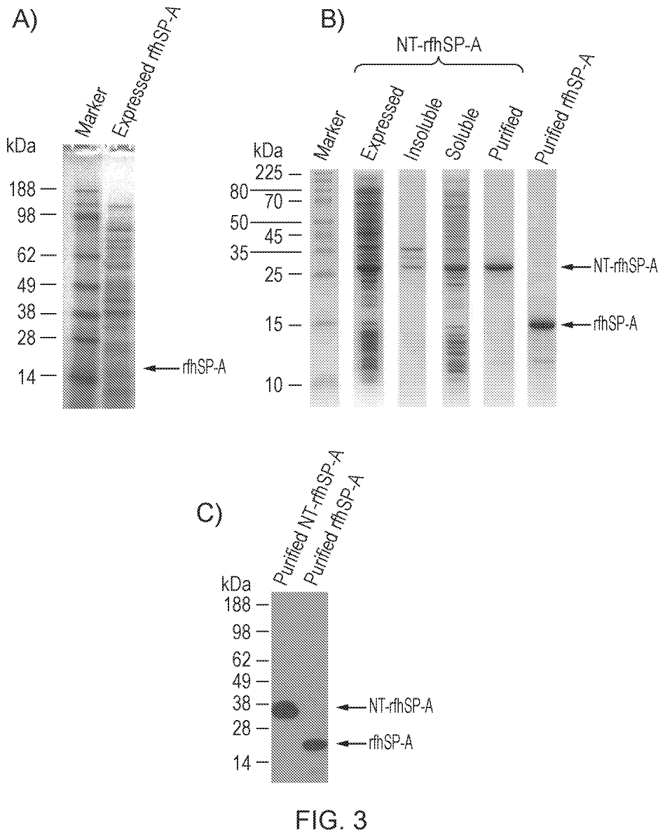

FIG. 3 shows the analysis of protein by reduced SDS-PAGE with subsequent Coomassie staining (FIGS. 3A and B) or western blot analysis using an antibody directed against native human SP-A (FIG. 3C). Expression of rfhSP-A alone was unable to be detected by Coomassie staining (FIG. 3A). However, high levels of expression were obtained upon expression of rfhSP-A as a fusion protein with the NT solubility tag. Upon lysis of E. coli, the majority of NT-rfhSP-A was in the soluble fraction (compare insoluble and soluble). This allowed for efficient purification of NT-rfhSP-A. Subsequent removal of the NT tag allowed purification of rfhSP-A (FIG. 3B). Protein identity was confirmed by western blot analysis (FIG. 3C).

Accordingly, the present method enables the expression of NT-rfhSP-A where rfh-SPA could not be expressed alone.

### Example 2—Expression and Purification of rfhSP-D Using a Solubility-Enhancing Moiety from the N-Terminal (NT) Fragment of a Spider Silk Protein

rfhSP-D with an N-terminal tag derived from spider silk protein (SEQ ID NO: 29) was successfully expressed in E. coli as a soluble protein and purified with subsequent cleavage of NT and purification of rfhSP-D.

SEQ ID NO: 29
MGHHHHHHMSHTTPWTNPGLAENFMNSFMQGLSSMPGFTASQLDKMST
IAQSMVQSIQSLAAQGRTSPNDLQALNMAFASSMAEIAASEEGGGSLS
TKTSSIASAMSNAFLQTTGVVNQPFINEITQLVSMFAQAGMNDVSAGN
SALEVLFQGPGLKGDKGIPGDKGAKGESGLPDVASLRQQVEALQGQVQ
HLQAAFSQYKKVELFPNGQSVGEKIFKTAGFVKPFTEAQLLCTQAGGQ
LASPRSAAENAALQQLVVAKNEAAFLSMTDSKTEGKFTYPTGESLVYS
NWAPGKPNDDGGSEDCVEIFTNGKWNDRACGEKRLVVCEF
Italic-His purification tag
Standard-Solubility enhancing moiety
Underline-cleavage site
Bold-rfhSP-D

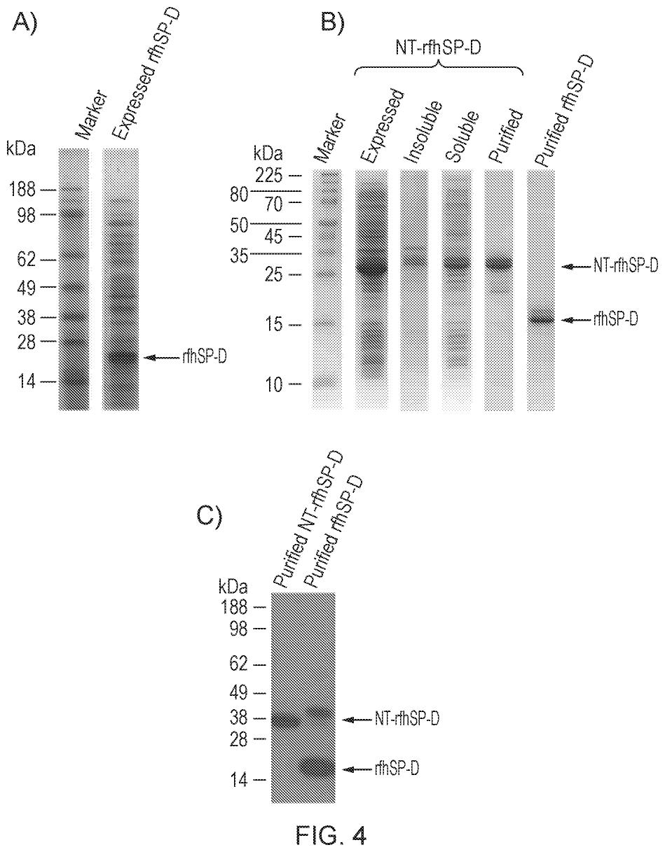

FIG. 4 shows analysis of protein by reduced SDS-PAGE with subsequent Coomassie staining (FIGS. 4A and B) or western blot analysis using an antibody directed against rfhSP-D (FIG. 4C). Expression of rfhSP-D alone gave expression levels which were detectable by Coomassie staining (FIG. 4A), however, higher levels of expression were obtained upon expression of rfhSP-D as a fusion protein with the NT solubility tag. Upon lysis of E. coli, the majority of NT-rfhSP-D was in the soluble fraction (compare insoluble and soluble). This allowed for efficient purification of NT-rfhSP-D. Subsequent removal of the NT tag allowed purification of rfhSP-D (FIG. 4B) The identity of rfhSP-D was confirmed by western blot analysis (FIG. 4C).

Comparison of the yields of SP-D generated by expression of full length SP-D, rfhSP-D fused to a maltose-binding protein (MBP) tag (following the method described by Hickling et al. (Eur J Immunol. 1999 November; 29(11):3478-84) and the present method are shown in Table 2 below.

TABLE 2

| Production method | Yield |
|---|---|
| Full length SP-D | 0.5-2 mg/litre |
| rfhSP-D and MBP | 5-10 mg/litre |
| fusion protein | |
| rfhSP-D with | 150 mg/litre |
| present NT tag | |

Accordingly, the present method vastly improved levels of expression of NT-rfhSP-D compared to the expression of rfhSP-D alone.

### Example 3—Gel Filtration and Affinity Purification of rfhSP-A and rfhSP-D

Gel filtration was undertaken to assess oligomeric state using a 24 ml superdex column equilibrated in 20 mM Tris 150 mM NaCl with 5 mM EDTA, pH 8.

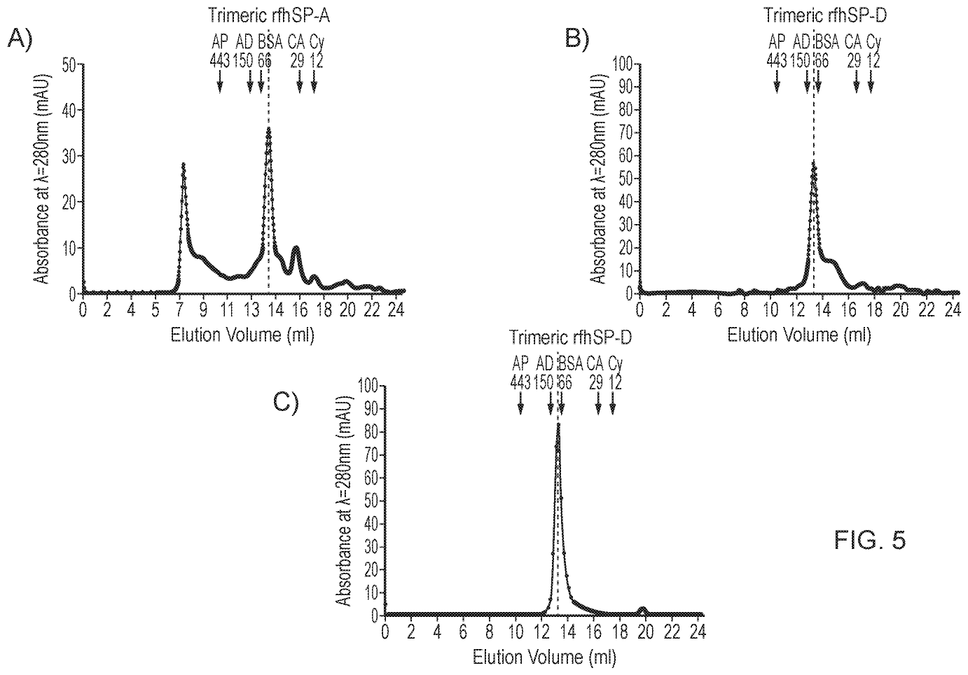

A large proportion of solubly expressed rfhSP-A was found to be trimeric with some being monomeric and dimeric and a proportion forming higher order aggregates (FIG. 5A). The majority of solubly expressed rfhSP-D was trimeric with a small fraction being lower molecular weight protein (FIG. 5B). Moreover, solubly expressed rfhSP-D at exactly the same elution volume as purified rfhSP-D produced previously by refolding (FIG. 5C). Trimeric rfhSP-A and rfhSP-D protein was isolated and used for further purification.

These results demonstrate that expression of rfhSP-A and rfhSP-D as soluble fusion proteins and subsequent purification enabled the production of trimeric rfhSP-A and rfhSP-D.

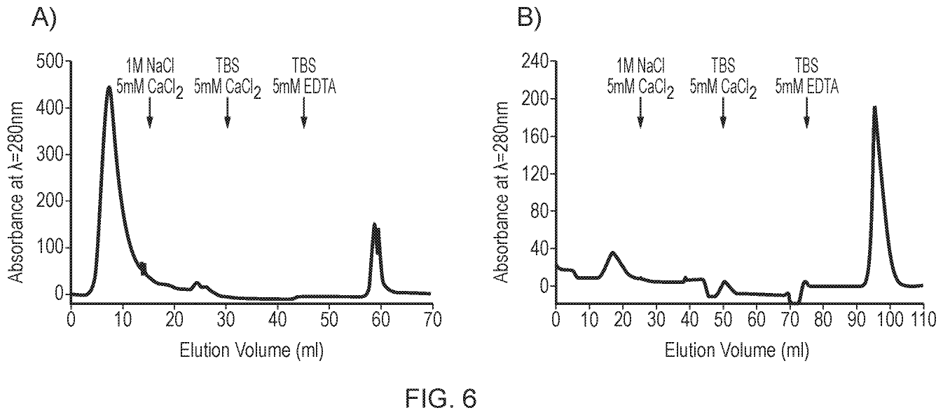

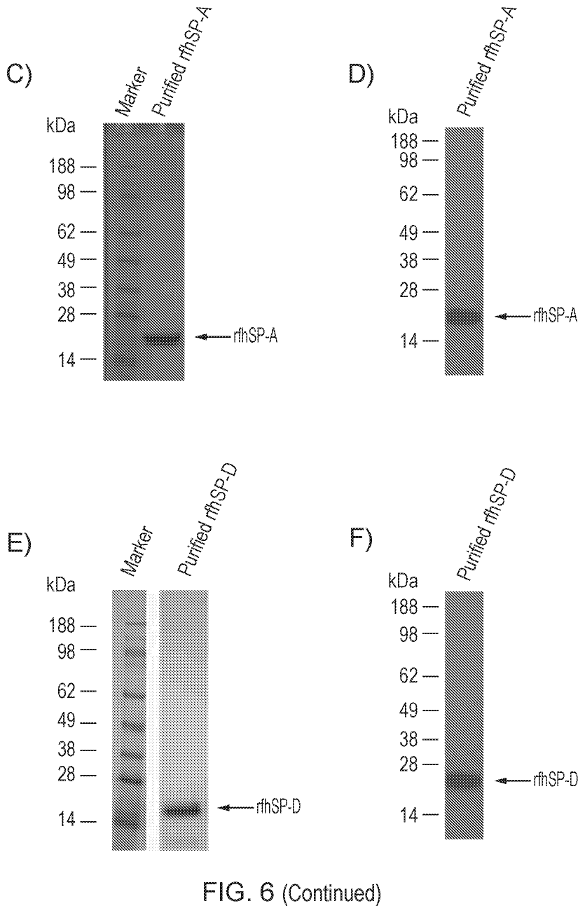

Affinity purified rfhSP-A and rfhSP-D resulted in generation of pure protein preparations. rfhSP-A and rfhSP-D were purified by mannan or ManNAc affinity purification, respectively. FIG. 6 shows chromatographs of the affinity purification of rfhSP-A (FIG. 6A) and rfhSP-D (FIG. 6B). Affinity columns were equilibrated in 20 mM Tris 150 mM NaCl, pH 8 (TBS) with 5 mM CaCl₂. After application of recombinant protein to the affinity columns, columns were washed with 1 column volume of TBS 5 mM CaCl₂, with subsequent washes in 1 M NaCl₂ followed again by TBS 5 mM CaCl₂. Bound recombinant rfhSP-A and rfhSP-D were eluted in TBS 5 mM EDTA. Eluted rfhSP-A and rfhSP-D was then analysed by reduced SDS-PAGE with subsequent Coomassie staining (FIGS. 6C and E, respectively) or western blot analysis using an antibody directed against native human SP-A or rfhSP-D (FIGS. 6D and F, respectively).

These results demonstrate that the trimeric rfhSP-A and trimeric rfhSP-D molecules were able to bind to a carbohydrate affinity column, allowing for efficient purification.

### Example 4—Binding of SP-A to Known Ligands

rfhSP-A was found to specifically bind to numerous ligands of natural human SP-A in the presence of calcium including mannan, klebsiella LPS and HIV protein gp120 IIIB.

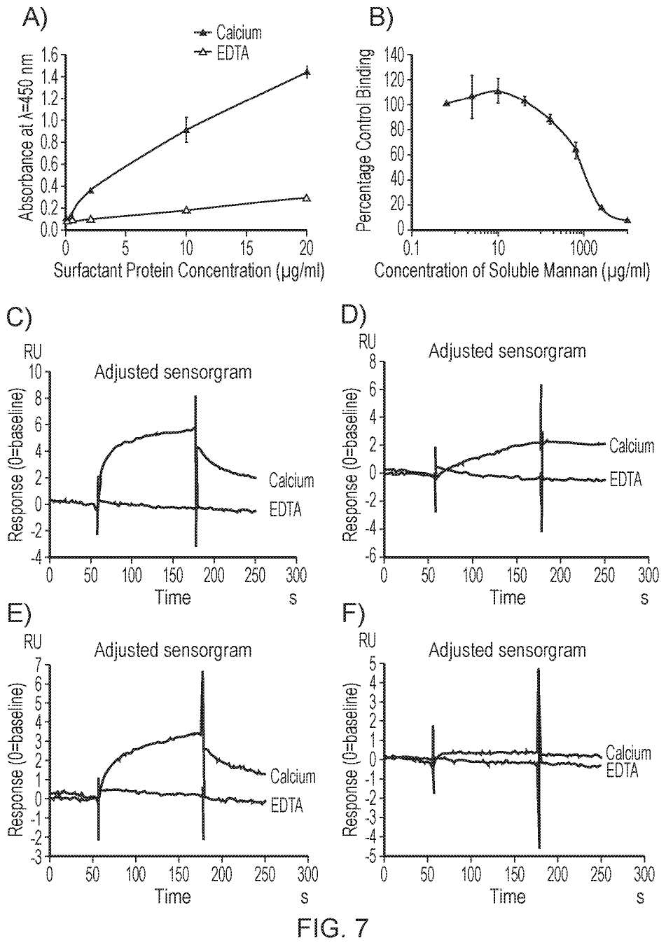

Increasing concentrations of rfhSP-A were added to ELISA plates coated in mannan (5 μg/well) in either the presence of 10 mM CaCl₂) or 50 mM EDTA (FIG. 7A). Levels of rfhSP-A were detected using an antibody directed against native human SP-A. Binding was shown to be specific by the inhibition of binding of rfhSP-A (5 μg/ml) upon addition of increasing concentrations of soluble mannan (FIG. 7B). rfhSP-A was also immobilized onto a Biacore sensor chip (to ˜700 RU), to which soluble ligands were found to bind specifically in the presence of calcium, including mannan (FIG. 7C), Klebsiella LPS (FIG. 7D) and HIV protein gp120 III B (FIG. 7E). A negative control of BSA was found not to be bound by the immobilised rhfSP-A (FIG. 7F).

### Example 5—Neutralisation of RSV by rfhSP-A

It was shown that rfhSP-A produced through use of an N-terminal tag derived from spider silk protein functions as an innate immune protein to neutralise RSV and prevent infection of human bronchial epithelial cells.

rfhSP-A was more effective at RSV neutralisation than native human SP-A. Dimeric rfhSP-A maintained some functionality in RSV neutralisation. However, the trimeric structure was essential for a fully functional neutralisation molecule.

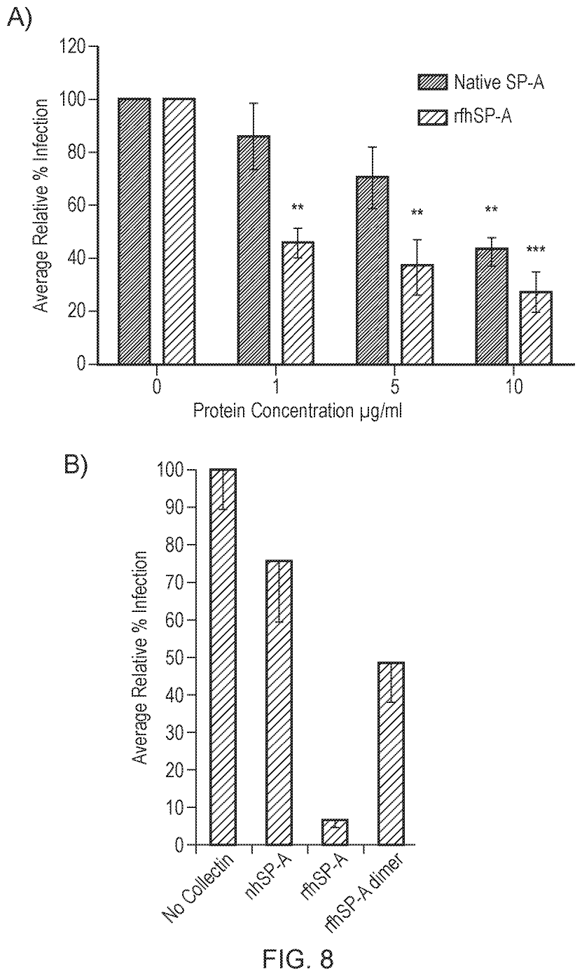

RSV was preincubated at 37° C. either alone or with increasing concentrations of native human SP-A or rfhSP-A for 1 hour. RSV containing SP-A was then put onto human bronchial epithelial cells (AALEB) and left for 2 hours a t 37° C. Cells were then washed and left to incubate for 24 hours. Levels or RSV infection were quantified using RT-qPCR. Results were normalised to the RSV alone (no SP-A) control. Shown is the mean (±standard error) of three experiments undertaken in duplicates (FIG. 8A).

This experiment was repeated at a concentration of 5 μg/ml with an additional control of purified dimeric rfhSP-A. However, quantification of % cells infected was undertaken by flow cytometry using an antibody directed against RSV F protein. Results were normalised to the RSV alone (no SP-A) control. Shown is the mean (±standard error) of one experiment undertaken in duplicates (FIG. 8B).

### Example 6—Binding of rfhSP-A to Antigen from Dermatophagoides pteronyssinus 1 and Lipopolysaccharide

Antigen from Dermatophagoides pteronyssinus 1 (DerP) (10 units/well), LPS from Haemophilus Influenzae (Eagan wildtype or Eagan 4A mutant) (1 μg/well) or BSA negative control (1 μg/well) was immobilised onto microtitre plates prior to adding varying concentrations of native human SP-A (nhSP-A) or rfhSP-A in 20 mM Tris-HCl, 150 mM NaCl, 5 mM CaCl₂ (TSC). Bound nhSP-A or rfhSP-A was detected with biotinylated rabbit polyclonal anti-SP-A (1:1000; Antibody Shop, Gentofte, Denmark), followed by the addition of streptavidin-HRP and tetramethylbenzene substrate with subsequent inhibition of reaction after 15 mins with 0.5 M H₂SO₄. Absorbance was measured at A=450 nm. Background binding to the BSA control was subtracted from the absorbance and means calculated (n=2).

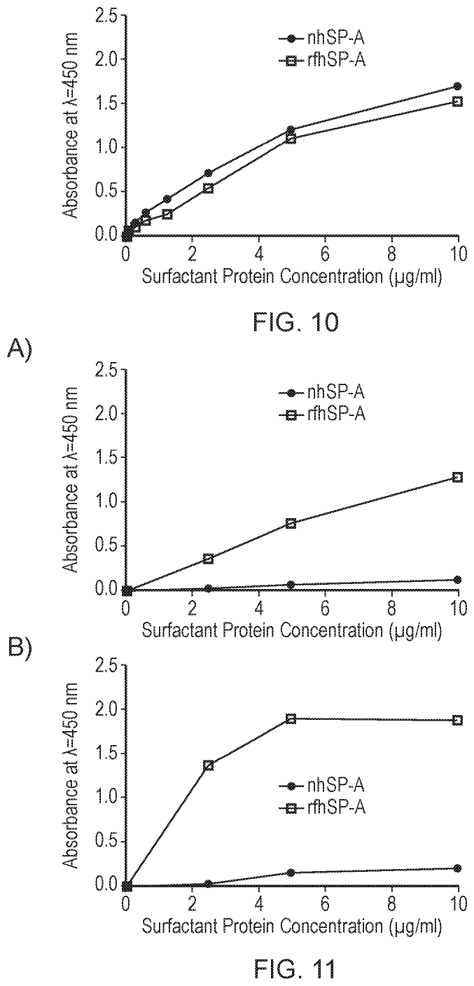

As shown in FIG. 10 rfhSP-A binds to DerP allergen from house dust mite to a similar degree as nhSP-A purified from human lung in the presence of calcium. This indicates that the CRD, neck and short collagen stalk is sufficient to allow binding and interaction with allergens.

As shown in FIG. 11 rfhSP-A binds to LPS from haemophilus influenza (Eagan wildtype and Eagan mutant 4A) in the presence of calcium. However, rfhSP-A binds to this LPS substantially more than nhSP-A. This correlates with the RSV data which shows rfhSP-A to be more effective at neutralising RSV than nhSP-A (FIG. 8).

As also shown in FIG. 11, rfhSP-A binds to the mutant 4A strain (which contains only one Heptose) of LPS more than the wildtype strain (FIG. 11B).

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A fusion protein comprising
i) a solubility-enhancing moiety which is derived from the N-terminal (NT) fragment of a spider silk protein; and
ii) a C-type lectin polypeptide.

2. A fusion protein according to claim 1 wherein the solubility-enhancing moiety comprises a sequence shown as SEQ ID NO: 1 to 14 or a variant thereof.

3. A fusion protein according to claim 2 wherein the solubility-enhancing moiety comprises a sequence shown as SEQ ID NO: 13 or a variant thereof.

4. A fusion protein according to any preceding claim wherein the C-type lectin is surfactant protein A (SP-A) or surfactant protein D (SP-D) or a fragment thereof, or a variant of the SP-A, SP-D or fragment thereof which has at least 70% sequence identity.

5. A fusion protein according to claim 4 wherein the C-type lectin is a truncated SP-A polypeptide which lacks at least amino acids 1 to 27 from the N-terminal of SEQ ID NO: 15 or a variant which has at least 70% sequence identity to a truncated SP-A polypeptide which lacks at least amino acids 1 to 27 from the N-terminal of SEQ ID NO: 15 and is capable of trimerisation.

6. A fusion protein according to claim 5 wherein the C-type lectin is a truncated SP-A polypeptide which lacks amino acids 1 to 30, 1 to 40, 1 to 50, 1 to 60 or 1 to 70 from the N-terminal of SEQ ID NO: 15 or a variant which has at least 70% sequence identity to a truncated SP-A polypeptide which lacks amino acids 1 to 30, 1 to 40, 1 to 50, 1 to 60 or 1 to 70 from the N-terminal of SEQ ID NO: 15 and is capable of trimerisation.

7. A fusion protein according to any of claims 1 to 6 wherein the C-type lectin is a truncated SP-A polypeptide which comprises the sequence shown in SEQ ID NO: 16, or a variant comprising an amino acid sequence having at least 70% sequence identity to the sequence shown as SEQ ID NO: 16.

8. A fusion protein according to any of claims 1 to 4 wherein the C-type lectin is a truncated SP-D polypeptide which lacks residues 1-178 from the N-terminal of the SEQ ID NO: 17 or a variant which has at least 70% sequence identity to a truncated SP-D polypeptide which substantially lacks residues 1-178 from the N-terminal of the SEQ ID NO: 17.

9. A fusion protein according to any of claims 1 to 4 wherein the C-type lectin is a truncated SP-D polypeptide which comprises the sequence shown in SEQ ID NO: 18, or a variant comprising an amino acid sequence having at least 70% sequence identity to the sequence shown as SEQ ID NO: 18

10. A fusion protein according to any preceding claim wherein the solubility-enhancing moiety is linked directly or indirectly to the amino-terminal of the C-type lectin polypeptide.

11. A fusion protein according to any preceding claim which comprises a cleavage site between the solubility-enhancing moiety and the C-type lectin polypeptide.

12. A fusion protein according to any preceding claim further comprising a purification tag.

13. A fusion protein according to any preceding claim which comprises the following structure:
Purification tag-solubility enhancing moiety-cleavage site-C-type lectin

14. A fusion protein according to claim 13 which comprises the sequence shown as SEQ ID NO: 28 or 29.

15. A nucleic acid sequence encoding a polypeptide according to any preceding claim.

16. A vector comprising a nucleic acid sequence according to claim 15

17. A method of producing a fusion protein according to any of claims 1 to 14 which comprises the steps of:
a) expressing a fusion protein as defined in any of claims 1 to 14 in a host cell;
b) obtaining a mixture including the fusion protein; and
c) optionally isolating the fusion protein.

18. A method according to claim 17, further comprising the step of isolating the C-type lectin polypeptide from the solubility-enhancing moiety.

19. A truncated SP-A polypeptide which lacks the N-terminal domain of full SP-A for use in treating or preventing a disease.

20. A truncated SP-A polypeptide for use according to claim 19 wherein the truncated SP-A polypeptide lacks at least amino acids 1 to 27 from the N-terminal of SEQ ID NO: 15 or a variant which has at least 70% sequence identity to a truncated SP-A polypeptide which lacks at least amino acids 1 to 27 from the N-terminal of SEQ ID NO: 15 and is capable of trimerisation.

21. A truncated SP-A polypeptide for use according to claim 19 or 20 wherein the truncated SP-A polypeptide lacks amino acids 1 to 30, 1 to 40, 1 to 50, 1 to 60 or 1 to 70 from the N-terminal of SEQ ID NO: 15 or a variant which has at least 70% sequence identity to a truncated SP-A polypeptide which lacks amino acids 1 to 30, 1 to 40, 1 to 50, 1 to 60 or 1 to 70 from the N-terminal of SEQ ID NO: 15 and is capable of trimerisation.

22. A truncated SP-A polypeptide for use according to any of claims claims 19 to 21 wherein the truncated SP-A polypeptide comprises SEQ ID NO: 16 or a variant which has at least 70% sequence identity thereto and is capable of trimerisation.

23. A truncated SP-A polypeptide for use according to any of claims claims 19 to 22 wherein the truncated SP-A polypeptide consists of SEQ ID NO: 16 or a variant which has at least 70% sequence identity thereto and is capable of trimerisation.

24. A method for treating or preventing a disease which comprises the step of administering a truncated SP-A polypeptide as defined in any of claims 19 to 23 to a subject.

25. Use of a truncated SP-A polypeptide as defined in any of claims 19 to 23 in the manufacture of a medicament for the treatment or prevention of a disease.

26. A use or method according to any of claims 19 to 25 wherein the disease is an inflammatory disease such as an infection or an allergy.

27. A use or method according to claim 26 wherein the disease is a viral, bacterial or fungal infection.

28. A use or method according to claim 26 wherein the disease is a viral infection.

29. A truncated SP-A polypeptide which lacks at least amino acids 1 to 27 from the N-terminal of SEQ ID NO: 15 or a variant which has at least 70% sequence identity thereto and is capable of trimerisation.

30. A truncated SP-A polypeptide as defined in any of claims 21 to 23.


SEQUENCE LISTING (36 sequences)

SEQ ID NO: 1 - protein, 131 aa, Euprosthenops australis
SHTTPWTNPGLAENFMNSFMQGLSSMPGFTASQLDDMSTIAQSMVQSIQSLAAQGRTSPNKLQALNMAFASSMAEIAASEEGGGSLSTKTSSIASAMSNAFLQTTGVVNQPFINEITQLVSMFAQAGMNDV

SEQ ID NO: 2 - protein, 127 aa, Latrodectus geometricus
QANTPWSSKQNADAFISAFMTAASQSGAFSSDQIDDMSVISNTLMAAMDNMGGRITPSKLQALDMAFASSVAEIAAVEGQNIGVTTNAISDALTSAFYQTTGVVNNKFISEIRSLINMFAQASANDV

SEQ ID NO: 3 - protein, 127 aa, Latrodectus hesperus
QANTPWSSKANADAFINSFISAASNTGSFSQDQMEDMSLIGNTLMAAMDNMGGRITPSKLQALDMAFASSVAEIAASEGGDLGVTTNAIADALTSAFYQTTGVVNSRFISEIRSLIGMFAQASANDV

SEQ ID NO: 4 - protein, 130 aa, Nephila clavipes
QNTPWSSTELADAFINAFMNEAGRTGAFTADQLDDMSTIGDTIKTAMDKMARSNKSSKGKLQALNMAFASSMAEIAAVEQGGLSVDAKTNAIADSLNSAFYQTTGAANPQFVNEIRSLINMFAQSSANEV

SEQ ID NO: 5 - protein, 131 aa, Argiope trifasciata
QGATPWENSQLAESFISRFLRFIGQSGAFSPNQLDDMSSIGDTLKTAIEKMAQSRKSSKSKLQALNMAFASSMAEIAVAEQGGLSLEAKTNAIASALSAAFLETTGYVNQQFVNEIKTLIFMIAQASSNEI

SEQ ID NO: 6 - protein, 124 aa, Latrodectus geometricus
LRWSSKDNADRFINAFLQAASNSGAFSSDQVDDMSVIGNTLMTAMDNMGGRITPSKLQALDMAFASSVAEIAVADGQNVGGATNAISNALRSAFYQTTGVVNNQFISEISNLINMFAQVSANEV

SEQ ID NO: 7 - protein, 127 aa, Latrodectus hesperus
QANTPWSSKENADAFIGAFMNAASQSGAFSSDQIDDMSVISNTLMAAMDNMGGRITQSKLQALDMAFASSVAEIAVADGQNVGAATNAISDALRSAFYQTTGVVNNQFITGISSLIGMFAQVSGNEV

SEQ ID NO: 8 - protein, 131 aa, Nephila inaurata madagascariensis
QANTPWSDTATADAFIQNFLGAVSGSGAFTPDQLDDMSTVGDTIMSAMDKMARSNKSSKSKLQALNMAFASSMAEIAAVEQGGQSMDVKTNAIANALDSAFYMTTGSTNQQFVNEMRSLINMLSAAAVNEV

SEQ ID NO: 9 - protein, 131 aa, Nephila clavipes
QARSPWSDTATADAFIQNFLAAVSGSGAFTSDQLDDMSTIGDTIMSAMDKMARSNKSSQHKLQALNMAFASSMAEIAAVEQGGMSMAVKTNAIVDGLNSAFYMTTGAANPQFVNEMRSLISMISAASANEV

SEQ ID NO: 10 - protein, 129 aa, Argiope bruennichi
AVPSVFSSPNLASGFLQCLTFGIGNSPAFPTQEQQDLDAIAQVILNAVSSNTGATASARAQALSTALASSLTDLLIAESAESNYSNQLSELTGILSDCFIQTTGSDNPAFVSRIQSLISVLSQNADTNI

SEQ ID NO: 11 - protein, 129 aa, Nephila clavata
PVPSVFSSPSLASGFLGCLTTGIGLSPAFPFQEQQDLDDLAKVILSAVTSNTDTSKSARAQALSTALASSLADLLISESSGSSYQTQISALTNILSDCFVTTTGSNNPAFVSRVQTLIGVLSQSSSNAI

SEQ ID NO: 12 - protein, 128 aa, Latrodectus hesperus
ASVNIFNSPNAATSFLNCLRSNIESSPAFPFQEQADLDSIAEVILSDVSSVNTASSATSLALSTALASSLAELLVTESAEEDIDNQVVALSTILSQCFVETTGSPNPAFVASVKSLLGVLSQSASNYE

SEQ ID NO: 13 - protein, 138 aa, Artificial Sequence
  solubility-enhancing moiety sequence
MSHTTPWTNPGLAENFMNSFMQGLSSMPGFTASQLDKMSTIAQSMVQSIQSLAAQGRTSPNDLQALNMAFASSMAEIAASEEGGGSLSTKTSSIASAMSNAFLQTTGVVNQPFINEITQLVSMFAQAGMNDVSAGNSA

SEQ ID NO: 14 - protein, 131 aa, Artificial Sequence
  consensus solubility-enhancing amino acid sequence
  MISC_FEATURE (20)..(20): Xaa may be Met or Leu
  MISC_FEATURE (23)..(23): Xaa may be Ala or Ile
  MISC_FEATURE (42)..(42): Xaa may be Asp, Asn or Gln
  MISC_FEATURE (50)..(50): Xaa may be Asn, Ser or Lys
  MISC_FEATURE (56)..(56): Xaa may be Lys or Arg
  MISC_FEATURE (84)..(84): Xaa may be Gly or Gln
  MISC_FEATURE (114)..(114): Xaa may be Asn or Ser
  MISC_FEATURE (121)..(121): Xaa may be Gly or Asn
  MISC_FEATURE (123)..(123): Xaa may be Phe or Leu
  MISC_FEATURE (124)..(124): Xaa may be Ala or Ser
QANTPWSSPNLADAFINSFXSAXSSSGAFSADQLDDMSTIGXTLMSAMDXMGRSGXSTKSKLQALNMAFASSMAEIAAAESGGXSVGVKTNAISDALSSAFYQTTGSVNPQFVXEIRSLIXMXXQASANEV

SEQ ID NO: 15 - protein, 248 aa, Homo sapiens
MWLCPLALNLILMAASGAVCEVKDVCVGSPGIPGTPGSHGLPGRDGRDGLKGDPGPPGPMGPPGEMPCPPGNDGLPGAPGIPGECGEKGEPGERGPPGLPAHLDEELQATLHDFRHQILQTRGALSLQGSIMTVGEKVFSSNGQSITFDAIQEACARAGGRIAVPRNPEENEAIASFVKKYNTYAYVGLTEGPSPGDFRYSDGTPVNYTNWYRGEPAGRGKEQCVEMYTDGQWNDRNCLYSRLTICEF

SEQ ID NO: 16 - protein, 171 aa, Artificial Sequence
  truncated SP-A polypeptide
GPGIPGECGEKGEPGERGPPGLPAHLDEELQATLHDFRHQILQTRGALSLQGSIMTVGEKVFSSNGQSITFDAIQEACARAGGRIAVPRNPEENEAIASFVKKYNTYAYVGLTEGPSPGDFRYSDGTPVNYTNWYRGEPAGRGKEQCVEMYTDGQWNDRNCLYSRLTICEF

SEQ ID NO: 17 - protein, 375 aa, Homo sapiens
MLLFLLSALVLLTQPLGYLEAEMKTYSHRTMPSACTLVMCSSVESGLPGRDGRDGREGPRGEKGDPGLPGAAGQAGMPGQAGPVGPKGDNGSVGEPGPKGDTGPSGPPGPPGVPGPAGREGALGKQGNIGPQGKPGPKGEAGPKGEVGAPGMQGSAGARGLAGPKGERGVPGERGVPGNTGAAGSAGAMGPQGSPGARGPPGLKGDKGIPGDKGAKGESGLPDVASLRQQVEALQGQVQHLQAAFSQYKKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDDGGSEDCVEIFTNGKWNDRACGEKRLVVCEF

SEQ ID NO: 18 - protein, 175 aa, Artificial Sequence
  truncated SP-D polypeptide
GPGLKGDKGIPGDKGAKGESGLPDVASLRQQVEALQGQVQHLQAAFSQYKKVELFPNGQSVGEKIFKTAGFVKPFTEAQLLCTQAGGQLASPRSAAENAALQQLVVAKNEAAFLSMTDSKTEGKFTYPTGESLVYSNWAPGKPNDDGGSEDCVEIFTNGKWNDRACGEKRLVVCE

SEQ ID NO: 19 - protein, 8 aa, Artificial Sequence
  HRV cleavage site
LEVLFQGP

SEQ ID NO: 20 - protein, 5 aa, Artificial Sequence
  EKT cleavage site
DDDDK

SEQ ID NO: 21 - protein, 4 aa, Artificial Sequence
  FXa cleavage site
IEGR

SEQ ID NO: 22 - protein, 7 aa, Artificial Sequence
  TEV cleavage site
ENLYFQG

SEQ ID NO: 23 - protein, 6 aa, Artificial Sequence
  Thr cleavage site
LVPRGS

SEQ ID NO: 24 - protein, 8 aa, Artificial Sequence
  histidine tag amino acid sequence
MGHHHHHH

SEQ ID NO: 25 - protein, 9 aa, Artificial Sequence
  hemagglutinin (HA) tag amino acid sequence
YPYDVPDYA

SEQ ID NO: 26 - protein, 9 aa, Artificial Sequence
  V5 epitope tag amino acid sequence
IPNPLLGLD

SEQ ID NO: 27 - protein, 10 aa, Artificial Sequence
  myc tag amino acid sequence
EQKLISEEDL

SEQ ID NO: 28 - protein, 323 aa, Artificial Sequence
  fusion protein
MGHHHHHHMSHTTPWTNPGLAENFMNSFMQGLSSMPGFTASQLDKMSTIAQSMVQSIQSLAAQGRTSPNDLQALNMAFASSMAEIAASEEGGGSLSTKTSSIASAMSNAFLQTTGVVNQPFINEITQLVSMFAQAGMNDVSAGNSALEVLFQGPGIPGECGEKGEPGERGPPGLPAHLDEELQATLHDFRHQILQTRGALSLQGSIMTVGEKVFSSNGQSITFDAIQEACARAGGRIAVPRNPEENEAIASFVKKYNTYAYVGLTEGPSPGDFRYSDGTPVNYTNWYRGEPAGRGKEQCVEMYTDGQWNDRNCLYSRLTICEF

SEQ ID NO: 29 - protein, 328 aa, Artificial Sequence
  fusion protein
MGHHHHHHMSHTTPWTNPGLAENFMNSFMQGLSSMPGFTASQLDKMSTIAQSMVQSIQSLAAQGRTSPNDLQALNMAFASSMAEIAASEEGGGSLSTKTSSIASAMSNAFLQTTGVVNQPFINEITQLVSMFAQAGMNDVSAGNSALEVLFQGPGLKGDKGIPGDKGAKGESGLPDVASLRQQVEALQGQVQHLQAAFSQYKKVELFPNGQSVGEKIFKTAGFVKPFTEAQLLCTQAGGQLASPRSAAENAALQQLVVAKNEAAFLSMTDSKTEGKFTYPTGESLVYSNWAPGKPNDDGGSEDCVEIFTNGKWNDRACGEKRLVVCEF

SEQ ID NO: 30 - protein, 119 aa, Homo sapiens
SIMTVGEKVFSSNGQSITFDAIQEACARAGGRIAVPRNPEENEAIASFVKKYNTYAYVGLTEGPSPGDFRYSDGTPVNYTNWYRGEPAGRGKEQCVEMYTDGQWNDRNCLYSRLTICEF

SEQ ID NO: 31 - protein, 29 aa, Homo sapiens
AHLDEELQATLHDFRHQILQTRGALSLQG

SEQ ID NO: 32 - protein, 23 aa, Artificial Sequence
  Gly-Xaa-Yaa motif sequence
GPGIPGECGEKGEPGERGPPGLP

SEQ ID NO: 33 - protein, 120 aa, Homo sapiens
NGQSVGEKIFKTAGFVKPFTEAQLLCTQAGGQLASPRSAAENAALQQLVVAKNEAAFLSMTDSKTEGKFTYPTGESLVYSNWAPGKPNDDGGSEDCVEIFTNGKWNDRACGEKRLVVCEF

SEQ ID NO: 34 - protein, 33 aa, Homo sapiens
DVASLRQQVEALQGQVQHLQAAFSQYKKVELFP

SEQ ID NO: 35 - protein, 23 aa, Artificial Sequence
  Gly-Xaa-Yaa motif sequence
GPGLKGDKGIPGDKGAKGESGLP

SEQ ID NO: 36 - protein, 14 aa, Artificial Sequence
  V5 epitope tag amino acid sequence
GKPIPNPLLGLDST